(19) United States
(12) Patent Application Publication
(10) Pub. No.: US 2025/0197336 A1
(43) Pub. Date: Jun. 19, 2025

(54) HYDROPHILIC EPOXY RESIN AND AQUEOUS COATING MATERIAL

(71) Applicant: INDUSTRIAL TECHNOLOGY RESEARCH INSTITUTE, Hsinchu (TW)

(72) Inventors: Chung-Han YU, Changhua City (TW); Ke-Hsuan LUO, Hsinchu City (TW); Po-Hsien HO, Changhua City (TW)

(73) Assignee: INDUSTRIAL TECHNOLOGY RESEARCH INSTITUTE, Hsinchu (TW)

(21) Appl. No.: 18/392,766

(22) Filed: Dec. 21, 2023

(30) Foreign Application Priority Data
Dec. 15, 2023 (TW) 112148928

(51) Int. Cl. C07C 51/09; C08G 59/38; C09D 5/00; C09D 7/20; C09D 163/00

(52) U.S. Cl. CPC C07C 51/09; C08G 59/38; C09D 5/00; C09D 7/20; C09D 163/00

(57) ABSTRACT

A hydrophilic epoxy resin and an aqueous coating material are provided. The hydrophilic epoxy resin is formed by reacting a multi-carboxylic acid compound with a bisphenol epoxy resin, wherein the multi-carboxylic acid compound is formed by reacting a first compound with an anhydride compound, and wherein the first compound is formed by reacting poly(ethylene glycol) with bis(2-hydroxyethyl) benzenedicarboxylate or dialkyl benzenedicarboxylate.

### CROSS-REFERENCE TO RELATED APPLICATION

The present application is based on, and claims priority from, Taiwan Application Serial Number 112148928, filed Dec. 15, 2023, the disclosure of which is hereby incorporated by reference herein in its entirety.

### TECHNICAL FIELD

The technical field relates to a hydrophilic epoxy resin.

### BACKGROUND

An epoxy resin coating is a high-performance coating that is used widely. It has several advantages such as having very strong adhesion to many kinds of substrate, high hardness of the coating layer, excellent water and chemical resistance, and good wear resistance, and can be used as floor coating, metal rust-resistant prime paint, marine coating, and the like. However, most of the present epoxy resin coating materials are solvent-based, and contain a large amount of volatile organic compound (VOC). VOC is poisonous, flammable, and pollutes the air, thereby being harmful to human health and the environment.

Environmental protection demand is increasing as science improves, and calls to use water-based coatings are getting louder day by day. The market for hydrophilic epoxy resin is expected to grow at a stable rate, and will likely exhibit a compound annual growth rate of nearly 8% during the forecast period, driving the prospects for continued growth of the global hydrophilic epoxy resin market. Early hydrophilic epoxy resins and emulsifiers are stirred at high speed in a homogenizer to prepare water-based coating materials. The water-based coating materials often have uneven emulsification, and the average particle size of the solid component (including the resin) is greater than 1000 nm, which can easily aggregate and precipitate, resulting in low storage stability of these water-based coating materials.

Accordingly, a novel hydrophilic epoxy resin with improved storage stability is called for.

### SUMMARY

One embodiment of the disclosure provides a hydrophilic epoxy resin formed by reacting a multi-carboxylic acid compound with a bisphenol epoxy resin, wherein the multi-carboxylic acid compound is formed by reacting a first compound with an anhydride compound, and wherein the first compound is formed by reacting poly(ethylene glycol) with bis(2-hydroxyethyl) benzenedicarboxylate or dialkyl benzenedicarboxylate, wherein the bis(2-hydroxyethyl) benzenedicarboxylate has a chemical structure of

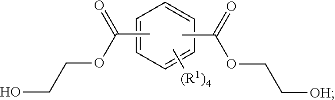

and wherein the dialkyl benzenedicarboxylate has a chemical structure of

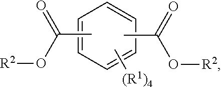

each of R¹ is independently H or C₁₋₄ alkyl group, and each of R² is independently C₁₋₄ alkyl group.

One embodiment of the disclosure provides an aqueous coating material, including 100 parts by weight of the described hydrophilic epoxy resin and 100 to 150 parts by weight of water.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a hydrophilic epoxy resin, which has self-emulsifying properties. For example, a method of forming the hydrophilic epoxy resin may include firstly reacting poly(ethylene glycol) with bis(2-hydroxyethyl) benzenedicarboxylate or dialkyl benzenedicarboxylate to form a first compound. The reaction is shown below:

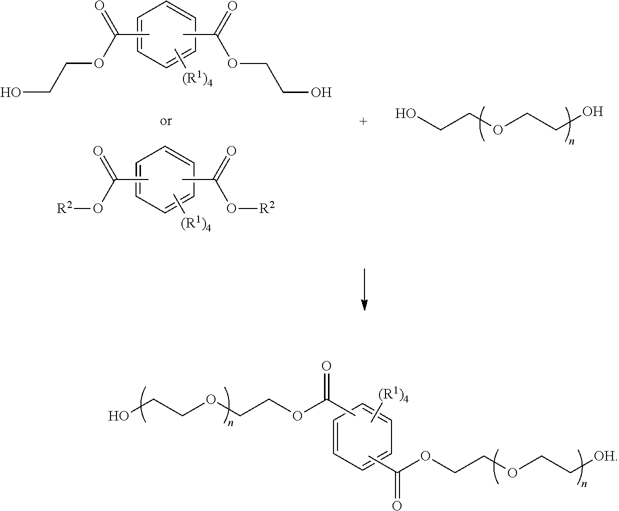

In the above formula, each of R¹ is independently H or C₁₋₄ alkyl group, each of R² is independently C₁₋₄ alkyl group, and n is determined by the weight average molecular weight of the poly(ethylene glycol), such as n is 10 to 70.

In some embodiments, the bis(2-hydroxyethyl) benzenedicarboxylate may include bis(2-hydroxyethyl) terephthalate (BHET).

In some embodiments, the dialkyl benzenedicarboxylate may include dimethyl benzenedicarboxylate. In some embodiments, the dimethyl benzenedicarboxylate may include dimethyl terephthalate (DMT).

In some embodiments, the poly(ethylene glycol) has a weight average molecular weight (Mw) of 400 to 3000. If the Mw of the poly(ethylene glycol) is too small, the hydrophilicity of the hydrophilic epoxy resin cannot be improved. If the Mw of the poly(ethylene glycol) is too large, the hydrophilicity of the hydrophilic epoxy resin cannot be further improved (or even worse).

Subsequently, the anhydride compound (such as trimellitic anhydride) is reacted with the first compound to form a multi-carboxylic acid compound. The reaction is shown below:

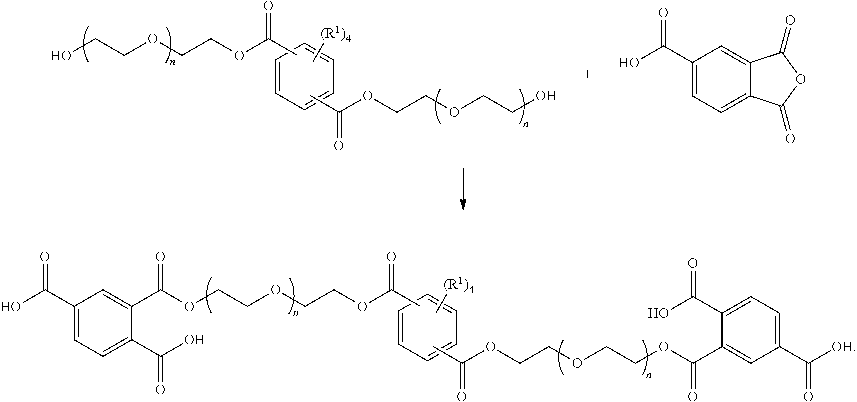

It should be understood that the trimellitic anhydride is used for illustration, and the anhydride compound can be another anhydride compound such as pyromellitic anhydride, 2,3,5-naphthalenetricarboxylic anhydride, 2,3,6-naphthalenetricarboxylic anhydride, 1,2,4-naphthalenetricarboxylic anhydride, 1,4,5,8-naphthalenetetracarboxylic dianhydride, 2,3,6,7-naphthalenetetracarboxylic dianhydride, 2,2′,3-biphenyltricarboxylic anhydride, 4,4′-oxydiphthalic anhydride, another suitable anhydride compound, or a combination thereof, as long as the formed multi-carboxylic acid compound has at least three carboxylic acid groups.

Subsequently, the multi-carboxylic acid compound is then reacted with bisphenol epoxy resin to form the hydrophilic epoxy resin. In some embodiments, the bisphenol epoxy resin may include bisphenol A epoxy resin, bisphenol B epoxy resin, bisphenol E epoxy resin, bisphenol F epoxy resin, bisphenol AF epoxy resin, bisphenol S epoxy resin, bisphenol fluorene epoxy resin, or a combination thereof. The reaction is shown below:

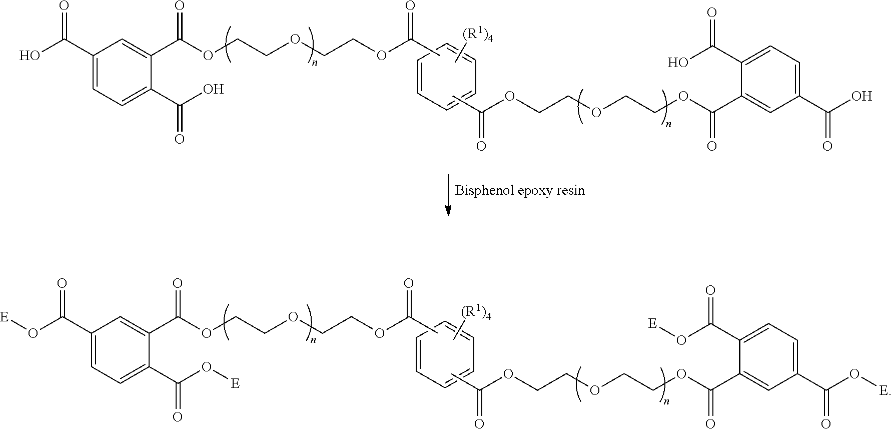

In the above formula, E is a residual group from the reaction of the carboxylic acid group of the multi-carboxylic acid compound with the epoxy group of the bisphenol epoxy resin. When the bisphenol epoxy resin is bisphenol A epoxy resin, E is

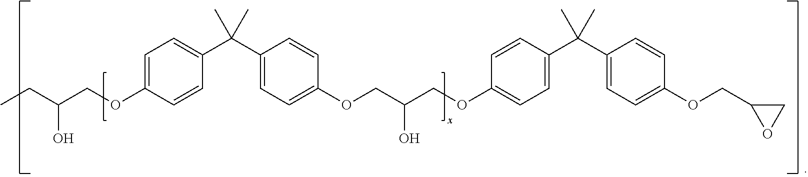

When the bisphenol epoxy resin is bisphenol B epoxy resin, E is

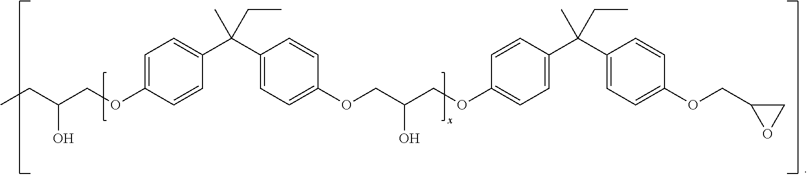

When the bisphenol epoxy resin is bisphenol E epoxy resin, E is

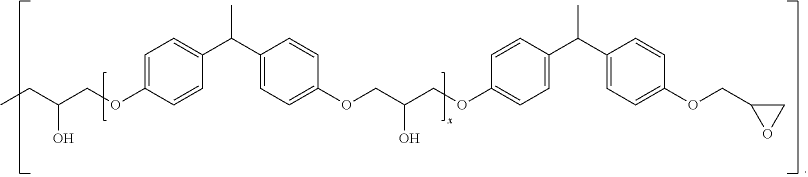

When the bisphenol epoxy resin is bisphenol F epoxy resin, E is

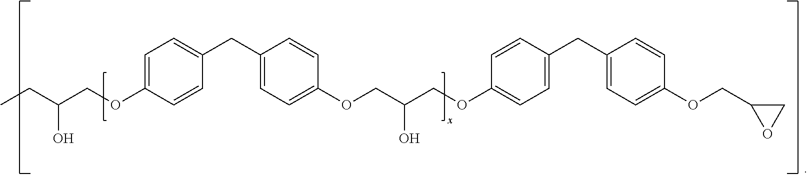

When the bisphenol epoxy resin is bisphenol AF epoxy resin, E is

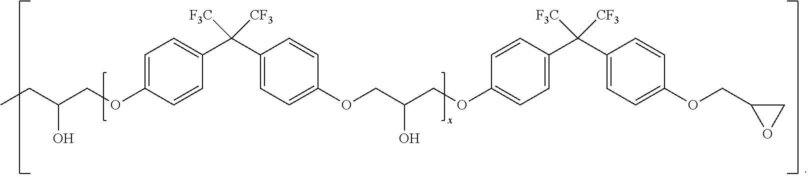

When the bisphenol epoxy resin is bisphenol S epoxy resin, E is

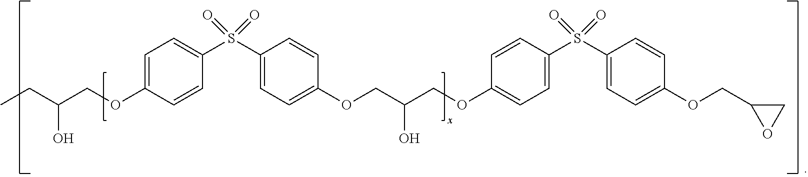

When the bisphenol epoxy resin is bisphenol fluorene epoxy resin, E is

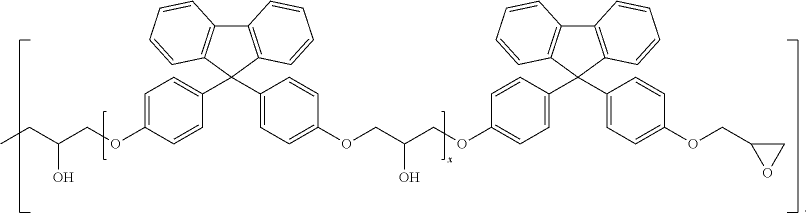

In the above formulae, x is determined by the epoxy equivalent of the bisphenol epoxy resin. For example, the bisphenol epoxy resin may have an epoxy equivalent of 170 to 1500. If the epoxy equivalent of the bisphenol epoxy resin is too high, the hydrophilicity of the hydrophilic epoxy resin cannot be further improved.

Note that the above reaction order is necessary, If the poly(ethylene glycol), the bis(2-hydroxyethyl) benzenedicarboxylate (or the dialkyl benzenedicarboxylate), the anhydride compound, and the bisphenol epoxy resin were mixed together to react, the product will be gelled and cannot form the hydrophilic epoxy resin.

The poly(ethylene glycol) having the hydrophilic groups and the aromatic dicarboxylic acid (ester) are introduced into the molecular structure of an epoxy resin to form the hydrophilic epoxy resin in the disclosure, such that the hydrophilic epoxy resin has a self-emulsifying effect. As such, there is no need to add an emulsifier such as poly(ethylene glycol) with a high molecular weight during preparing the coating material. In addition, the anhydride compound having at least three carboxylic acid groups can be introduced into the molecular structure, thereby preventing the coating material from curing and yellowing during storage. Furthermore, the hydrophilic epoxy resin of the disclosure has multiple epoxy groups, which help network crosslink structures formed quickly during the curing process, enhancing the curing efficiency.

Because the hydrophilic epoxy resin in some embodiments of the disclosure has self-emulsifying properties, there is no need to add an emulsifier. When water and hydrophilic epoxy resin are mixed, the mixture can directly emulsify and form a coating material.

One embodiment of the disclosure provides an aqueous coating material, including 100 parts by weight of the described hydrophilic epoxy resin and 100 to 150 parts by weight of water. If there is not enough water, this will lead to problems such as incomplete aquation, an emulsion viscosity that is too high, or layers formed in the coating material. If there is too much water, the properties will not be influenced much, but the aqueous coating material will have a solid content that is too low, which is a disadvantage for coating formulation.

In some embodiments, the aqueous coating material may further include 10 to 50 parts by weight of an additional bisphenol epoxy resin, based on 100 parts by weight of the described hydrophilic epoxy resin. The additional bisphenol epoxy resin can be the same as or different from the bisphenol epoxy resin used to form the hydrophilic epoxy resin. The additional bisphenol epoxy resin may further enhance the hardness of the coating layer formed from the aqueous coating material. If there is too much additional bisphenol epoxy resin, the storage stability of the aqueous coating material may be lowered.

In some embodiments, the aqueous coating material may further include 1 to 20 parts by weight of an additive, based on 100 parts by weight of the described hydrophilic epoxy resin. For example, the additive may include a wetting agent, a colorant, a thickener, a leveling agent, a co-solvent, or a combination thereof. The additive may improve properties of the aqueous coating material such as coatability, appearance, and storage stability. If too much additive is added, the physical and chemical properties of the aqueous coating material will be negatively influenced.

The aqueous coating material may have a solid content of 40% to 55%, and the average diameter of the solid component (the resin) is less than 1000 nm (or even less than 500 nm), which allows the coating material to be stored for a long time. The aqueous coating material can be stored alone. The aqueous coating material and a curing agent (or an aqueous solution of the curing agent) can be mixed further before coating. The mixture is then coated to form a layer. The cured coating layer may have a hardness of 2H (or even 3H).

Below, exemplary embodiments are described in detail so as to be easily realized by a person having ordinary knowledge in the art. The inventive concept may be embodied in various forms without being limited to the exemplary embodiments set forth herein. Descriptions of well-known parts are omitted for clarity.

### EXAMPLES

### Example 1

100 g of the poly(ethylene glycol) having Mw of 2000 was dried at 120° C. under reduced pressure for 1 hour. Normal pressure is then returned by introducing nitrogen. 6.4 g of bis(2-hydroxyethyl) terephthalate (BHET) and 0.04 g of tetrabutyl titanate serving as catalyst were added to poly(ethylene glycol) and mixed. The mixture was heated from 120° C. to 260° C., and then vacuumed to react for 6 hours to form a first compound. Subsequently, the first compound was cooled down to 100° C., and 0.5 g of anti-oxidant IRGANOX 1010 (commercially available from Double Bond Chemical Ind. Co., Ltd.) was added to the first compound. The reaction is shown below:

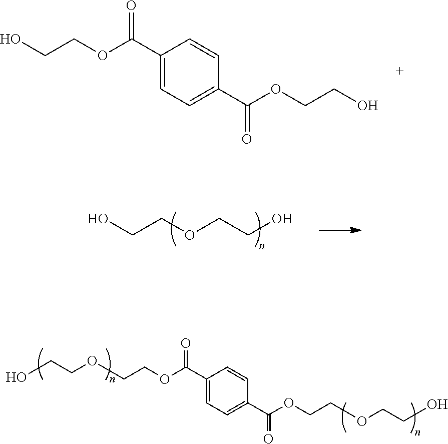

Subsequently, 9.77 g of trimellitic anhydride and 0.48 g of triphenylphosphine were added to 100 g of the mixture of the first compound and the anti-oxidant at 100° C. The mixture was then heated from 100° C. to 150° C., and then reacted under nitrogen for 6 hours to form a multi-carboxylic acid compound. The reaction is shown below:

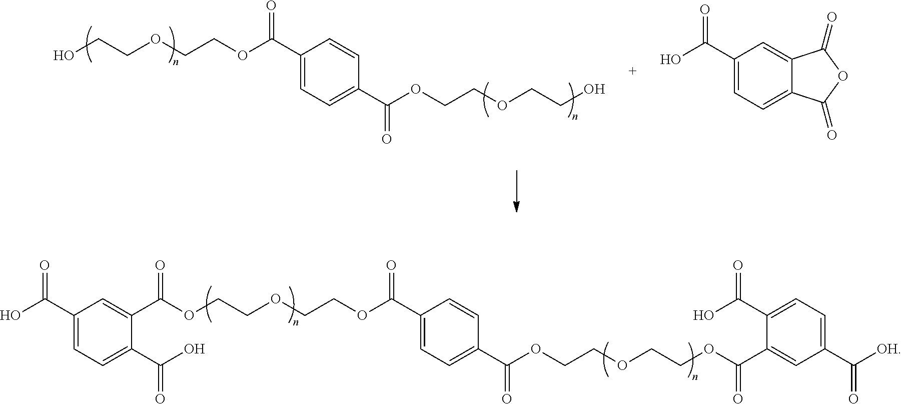

The multi-carboxylic acid compound was cooled down to 100° C., and 427 g of bisphenol A epoxy resin BE188 and 0.22 g of triphenylphosphine were added to the multi-carboxylic acid compound. The mixture was then heated from 100° C. to 120° C. and reacted for 3 hours under nitrogen, thereby obtaining a hydrophilic epoxy resin S1. The reaction is shown below:

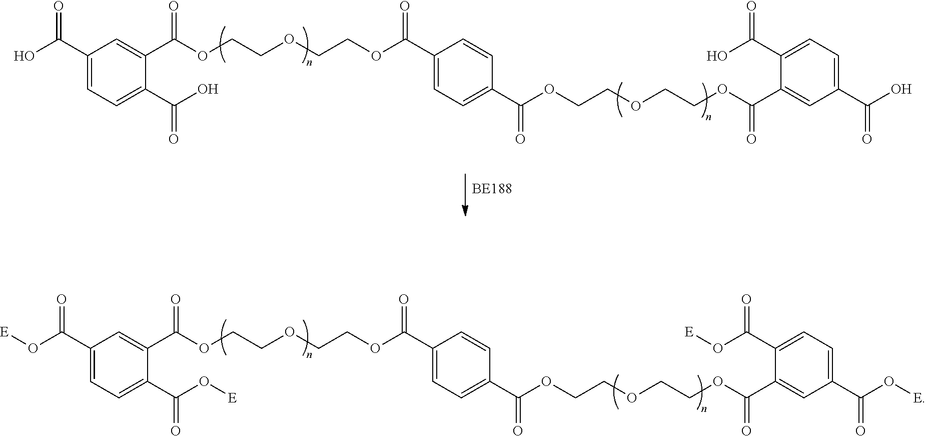

In the above formula, E is a residual group from the reaction of the carboxylic acid group of the multi-carboxylic acid compound with the epoxy group of the bisphenol A epoxy resin BE188. BE188 had an epoxy equivalent of 182 to 192. For example, E is

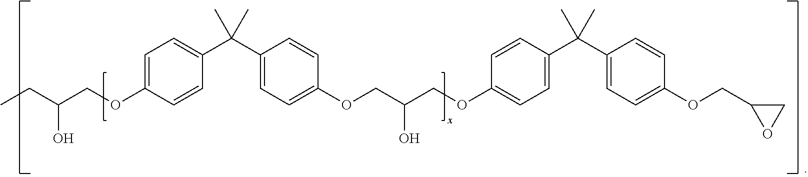

50 g of the hydrophilic epoxy resin S1 was stirred, and 50 g of water was then slowly added into the hydrophilic epoxy resin S1 to form an aqueous coating material P1 (with a solid content of 50%). The aqueous coating material P1 was analyzed by a dynamic light scattering instrument, and the hydrophilic epoxy resin S1 in the coating material had a diameter of 230 nm. The aqueous coating material P1 had a storage stability at 50° C. of longer than 21 days (measured according to the standard ASTM D1849). 29.6 g of a curing agent BECKOPOX EH 613w/80WA (commercially available from Allnex) was dissolved in 13.7 g of water, and then mixed with 100 g of the aqueous coating material P1 and form a coating layer that was then cured. The cured coating layer had a hardness of 2H (measured according to the standard ASTM D3363).

### Example 2

100 g of the poly(ethylene glycol) having Mw of 2000 was dried at 120° C. under reduced pressure for 1 hour. Normal pressure is then returned by introducing nitrogen. 4.8 g of dimethyl terephthalate (DMT) and 0.12 g of tetrabutyl titanate serving as catalyst were added to poly(ethylene glycol) and mixed. The mixture was heated from 120° C. to 260° C., and then vacuumed to react for 6 hours to form a first compound. Subsequently, the first compound was cooled down to 100° C., and 0.5 g of anti-oxidant IRGANOX 1010 (commercially available from Double Bond Chemical Ind. Co., Ltd.) was added to the first compound. The reaction is shown below:

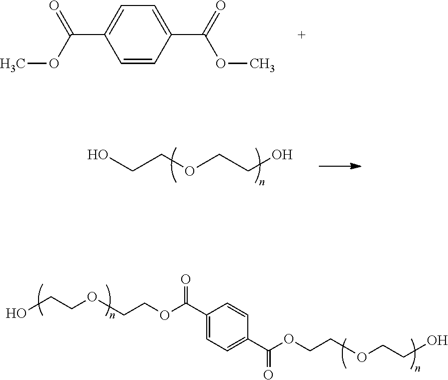

Subsequently, 9.77 g of trimellitic anhydride and 0.48 g of triphenylphosphine were added to 100 g of the mixture of the first compound and the anti-oxidant at 100° C. The mixture was then heated from 100° C. to 150° C., and then reacted under nitrogen for 6 hours to form a multi-carboxylic acid compound. The reaction is shown below:

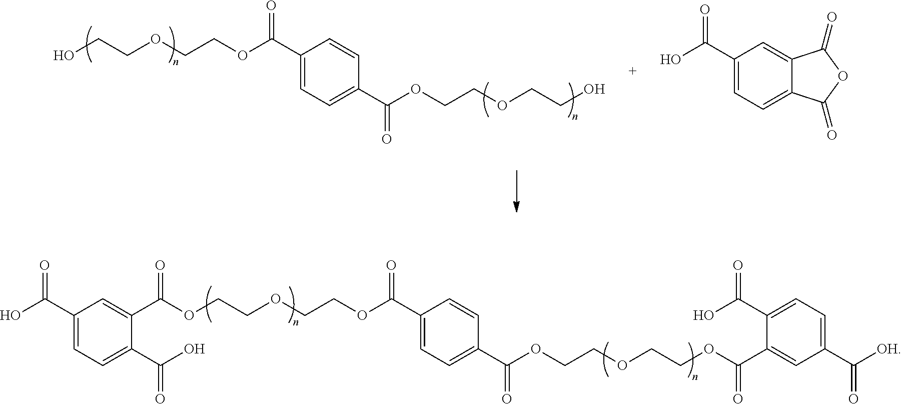

The multi-carboxylic acid compound was cooled down to 100° C., and 427 g of bisphenol A epoxy resin BE188 and 0.22 g of triphenylphosphine were added to the multi-carboxylic acid compound. The mixture was then heated from 100° C. to 120° C. and reacted for 3 hours under nitrogen, thereby obtaining a hydrophilic epoxy resin S2. The reaction is shown below:

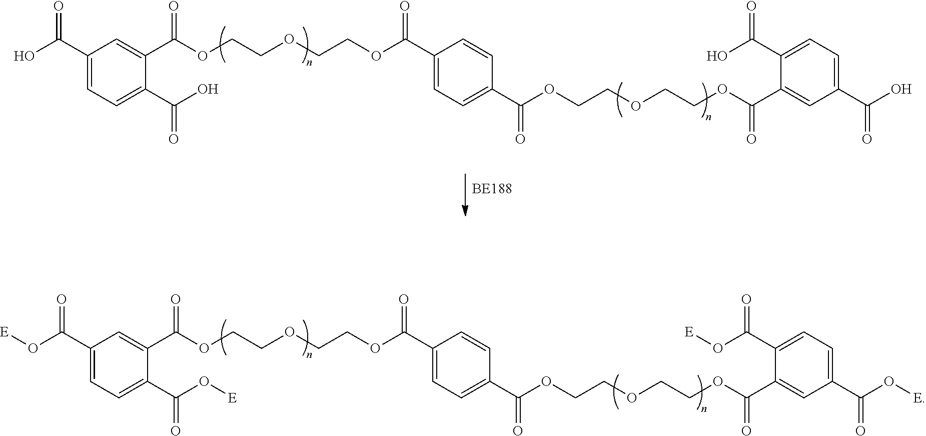

In the above formula, E is a residual group from the reaction of the carboxylic acid group of the multi-carboxylic acid compound with the epoxy group of the bisphenol A epoxy resin BE188. BE188 had an epoxy equivalent of 182 to 192. For example, E is

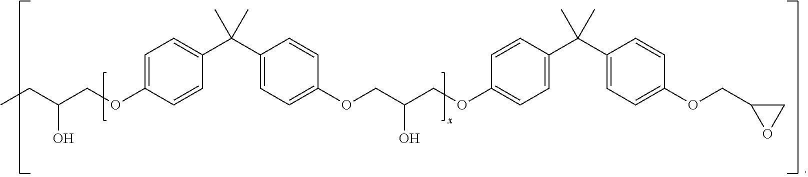

50 g of the hydrophilic epoxy resin S2 was stirred, and 50 g of water was then slowly added into the hydrophilic epoxy resin S2 to form an aqueous coating material P2 (with a solid content of 50%). The aqueous coating material P2 was analyzed by a dynamic light scattering instrument, and the hydrophilic epoxy resin S2 in the coating material had a diameter of 240 nm. The aqueous coating material P2 had a storage stability at 50° C. of longer than 21 days (measured according to the standard ASTM D1849). 29.6 g of the curing agent BECKOPOX EH 613w/80WA (commercially available from Allnex) was dissolved in 13.7 g of water, and then mixed with 100 g of the aqueous coating material P2 and form a coating layer that was then cured. The cured coating layer had a hardness of 2H (measured according to the standard ASTM D3363).

### Example 3

100 g of the poly(ethylene glycol) having Mw of 2000 was dried at 120° C. under reduced pressure for 1 hour. Normal pressure is then returned by introducing nitrogen. 6.4 g of BHET and 0.04 g of tetrabutyl titanate serving as catalyst were added to poly(ethylene glycol) and mixed. The mixture was heated from 120° C. to 260° C., and then vacuumed to react for 6 hours to form a first compound. Subsequently, the first compound was cooled down to 100° C., and 0.5 g of anti-oxidant IRGANOX 1010 (commercially available from Double Bond Chemical Ind. Co., Ltd.) was added to the first compound.

Subsequently, 9.77 g of trimellitic anhydride and 0.48 g of triphenylphosphine were added to 100 g of the mixture of the first compound and the anti-oxidant at 100° C. The mixture was then heated from 100° C. to 150° C., and then reacted under nitrogen for 6 hours to form a multi-carboxylic acid compound.

The multi-carboxylic acid compound was cooled down to 100° C., and 633 g of bisphenol A epoxy resin BE188 and 0.22 g of triphenylphosphine were added to the multi-carboxylic acid compound. The mixture was then heated from 100° C. to 120° C. and reacted for 3 hours under nitrogen, thereby obtaining a hydrophilic epoxy resin S3.

50 g of the hydrophilic epoxy resin S3 was stirred, and 50 g of water was then slowly added into the hydrophilic epoxy resin S3 to form an aqueous coating material P3 (with a solid content of 50%). The aqueous coating material P3 was analyzed by a dynamic light scattering instrument, and the hydrophilic epoxy resin S3 in the coating material had a diameter of 360 nm. The aqueous coating material P3 had a storage stability at 50° C. of longer than 21 days (measured according to the standard ASTM D1849). 32.2 g of the curing agent BECKOPOX EH 613w/80WA (commercially available from Allnex) was dissolved in 13.7 g of water, and then mixed with 100 g of the aqueous coating material P3 and form a coating layer that was then cured. The cured coating layer had a hardness of 3H (measured according to the standard ASTM D3363).

### Example 4

100 g of the poly(ethylene glycol) having Mw of 2000 was dried at 120° C. under reduced pressure for 1 hour. Normal pressure is then returned by introducing nitrogen. 6.4 g of BHET and 0.04 g of tetrabutyl titanate serving as catalyst were added to poly(ethylene glycol) and mixed. The mixture was heated from 120° C. to 260° C., and then vacuumed to react for 6 hours to form a first compound. Subsequently, the first compound was cooled down to 100° C., and 0.5 g of anti-oxidant IRGANOX 1010 (commercially available from Double Bond Chemical Ind. Co., Ltd.) was added to the first compound.

Subsequently, 9.77 g of trimellitic anhydride and 0.48 g of triphenylphosphine were added to 100 g of the mixture of the first compound and the anti-oxidant at 100° C. The mixture was then heated from 100° C. to 150° C., and then reacted under nitrogen for 6 hours to form a multi-carboxylic acid compound.

The multi-carboxylic acid compound was cooled down to 100° C., and 666 g of bisphenol A epoxy resin NPES 902 (having an epoxy equivalent of 600 to 650) and 0.22 g of triphenylphosphine were added to the multi-carboxylic acid compound. The mixture was then heated from 100° C. to 120° C. and reacted for 3 hours under nitrogen, thereby obtaining a hydrophilic epoxy resin S4.

50 g of the hydrophilic epoxy resin S4 was stirred, and 50 g of water was then slowly added into the hydrophilic epoxy resin S4 to form an aqueous coating material P4 (with a solid content of 50%). The aqueous coating material P4 was analyzed by a dynamic light scattering instrument, and the hydrophilic epoxy resin S4 in the coating material had a diameter of 400 nm. The aqueous coating material P4 had a storage stability at 50° C. of longer than 21 days (measured according to the standard ASTM D1849). 12.2 g of the curing agent BECKOPOX EH 613w/80WA (commercially available from Allnex) was dissolved in 13.7 g of water, and then mixed with 100 g of the aqueous coating material P4 and form a coating layer that was then cured. The cured coating layer had a hardness of 3H (measured according to the standard ASTM D3363).

### Comparative Example 1

100 g of the poly(ethylene glycol) having Mw of 2000 was dried at 120° C. under reduced pressure for 1 hour. Subsequently, 19 g of trimellitic anhydride and 0.48 g of triphenylphosphine were added to 100 g of poly(ethylene glycol) at 100° C. The mixture was then heated from 100° C. to 150° C., and then reacted under nitrogen for 6 hours to form a multi-carboxylic acid compound. The reaction is shown below:

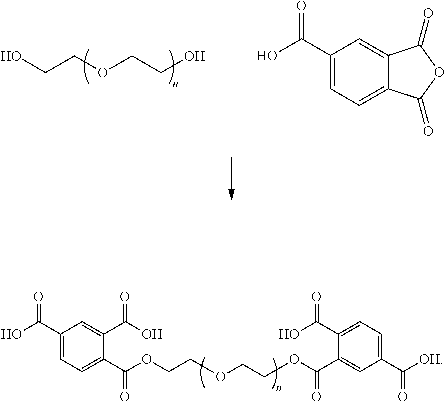

The multi-carboxylic acid compound was cooled down to 100° C., and 50 g of bisphenol A epoxy resin BE188 and 0.22 g of triphenylphosphine were added to the multi-carboxylic acid compound. The mixture was then heated from 100° C. to 120° C. and reacted for 3 hours under nitrogen, thereby obtaining a hydrophilic epoxy resin C1. The reaction is shown below:

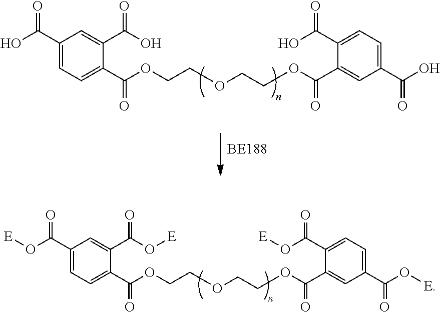

In the above formula, E is a residual group from the reaction of the carboxylic acid group of the multi-carboxylic acid compound with the epoxy group of the bisphenol A epoxy resin BE188, such as the chemical structure of E in Example 1.

50 g of the hydrophilic epoxy resin C1 was stirred, and 50 g of water was then slowly added into the hydrophilic epoxy resin C1 to form an aqueous coating material C2 (with a solid content of 50%). The aqueous coating material C2 was analyzed by a dynamic light scattering instrument, and the hydrophilic epoxy resin C1 in the coating material had a diameter of greater than 1000 nm. The aqueous coating material C2 had a storage stability at 50° C. of shorter than 2 days (measured according to the standard ASTM D1849). Accordingly, the hydrophilic epoxy resin lacking the aromatic core had poor storage stability.

### Comparative Example 2

100 g of poly(ethylene glycol) having Mw of 2000 was dried at 120° C. under reduced pressure for 1 hour. Normal pressure is then returned by introducing nitrogen. 6.4 g of bis(2-hydroxyethyl) terephthalate (BHET), 0.04 g of tetrabutyl titanate serving as catalyst, 9.77 g of trimellitic anhydride, 0.7 g of triphenylphosphine, and 427 g of bisphenol A epoxy resin BE188 were added to poly(ethylene glycol) and mixed. The mixture was heated from 120° C. to 260° C. under nitrogen to react for 1 hour. The viscosity of the mixture increased rapidly and then gelled and could not be used.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A hydrophilic epoxy resin, being
formed by reacting a multi-carboxylic acid compound with a bisphenol epoxy resin,
wherein the multi-carboxylic acid compound is formed by reacting a first compound with an anhydride compound, and
wherein the first compound is formed by reacting poly(ethylene glycol) with bis(2-hydroxyethyl) benzenedicarboxylate or dialkyl benzenedicarboxylate,
wherein the bis(2-hydroxyethyl)benzenedicarboxylate has a chemical structure of 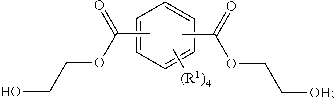
and
wherein the dialkyl benzenedicarboxylate has a chemical structure of 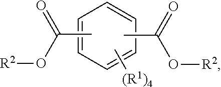
wherein each of R¹ is independently H or C₁₋₄ alkyl group, and each of R² is independently C₁₋₄ alkyl group.

2. The hydrophilic epoxy resin as claimed in claim 1, wherein the bis(2-hydroxyethyl) benzenedicarboxylate comprises bis(2-hydroxyethyl) terephthalate.

3. The hydrophilic epoxy resin as claimed in claim 1, wherein the dialkyl benzene dicarboxylate comprises dimethyl benzene dicarboxylate.

4. The hydrophilic epoxy resin as claimed in claim 1, wherein the poly(ethylene glycol) has a weight average molecular weight of 400 to 3000.

5. The hydrophilic epoxy resin as claimed in claim 1, wherein the anhydride compound comprises trimellitic anhydride, pyromellitic anhydride, 2,3,5-naphthalenetricarboxylic anhydride, 2,3,6-naphthalenetricarboxylic anhydride, 1,2,4-naphthalenetricarboxylic anhydride, 1,4,5,8-naphthalenetetracarboxylic dianhydride, 2,3,6,7-naphthalenetetracarboxylic dianhydride, 2,2′,3-biphenyltricarboxylic anhydride, 4,4′-oxydiphthalic anhydride, or a combination thereof.

6. The hydrophilic epoxy resin as claimed in claim 1, wherein the bisphenol epoxy resin has an epoxy equivalent of 170 to 1500.

7. The hydrophilic epoxy resin as claimed in claim 1, wherein the bisphenol epoxy resin comprises bisphenol A epoxy resin, bisphenol B epoxy resin, bisphenol E epoxy resin, bisphenol F epoxy resin, bisphenol AF epoxy resin, bisphenol S epoxy resin, bisphenol fluorene epoxy resin, or a combination thereof.

8. An aqueous coating material, comprising:
100 parts by weight of the hydrophilic epoxy resin as claimed in claim 1; and
100 to 150 parts by weight of water.

9. The aqueous coating material as claimed in claim 8, further comprising 10 to 50 parts by weight of an additional bisphenol epoxy resin.

10. The aqueous coating material as claimed in claim 8, further comprising 1 to 20 parts by weight of an additive.

11. The aqueous coating material as claimed in claim 10, wherein the additive comprises a wetting agent, a colorant, a thickener, a leveling agent, a co-solvent, or a combination thereof.
